SEQUENCE LISTING (1 sequence)

SEQ ID NO: 1 - protein, 556 aa, Artificial Sequence
  Recombinant PPE2 protein
MTAPIWMASPPEVHSALLSSGPGPGPLLVSAEGWHSLSIAYAETADELAALLAAVQAGTWDGPTAAVYVAAHTPYLAWLVQASANSAAMATRQETAATAYGTALAAMPTLAELGANHALHGVLMATNFFGINTIPIALNESDYARMWIQAATTMASYQAVSTAAVAAAPQTTPAPQIVKANAPTAASDEPNQVQEWLQWLQKIGYTDFYNNVIQPFINWLTNLPFLQAMFSGFDPWLPSLGNPLTFLSPANIAFALGYPMDIGSYVAFLSQTFAFIGADLAAAFASGNPATIAFTLMFTTVEAIGTIITDTIALVKTLLEQTLALLPAALPLLAAPLAPLTLAPASAAGGFAGLSGLAGLVGIPPSAPPVIPPVAAIAPSIPTPTPTPAPAPAPTAVTAPTPPPGPPPPPVTAPPPVTGAGIQSFGYLVGDLNSAAQARKAVGTGVRKKTPEPDSAEAPAAAAAPEEQVQPQRRRRPKIKQLGRGYEYLDLDPETGHDPTGSPQGAGTLGFAGTTHKASPGQVAGLITLPNDAFGGSPRTPMMPGTWDTDSATRVE

(19) United States
(12) Patent Application Publication
(10) Pub. No.: US 2020/0215151 A1
(43) Pub. Date: Jul. 9, 2020

(54) THERAPEUTIC COMPOSITION FOR INFLAMMATION/TISSUE INJURY

(71) Applicant: Centre for DNA Fingerprinting and Diagnostics (CDFD), Hyderabad (IN)

(72) Inventors: Sangita Mukhopadhyay, Hyderabad (IN); Ravi Pal, Hyderabad (IN); Madhu Babu Battu, Hyderabad (IN)

(21) Appl. No.: 16/737,012

(22) Filed: Jan. 8, 2020

(30) Foreign Application Priority Data
Jan. 8, 2019 (IN) 201941000876

(51) Int. Cl. A61K 38/16; C07K 14/35; A61K 9/06; A61K 47/02; A61K 47/12; A61K 47/10; A61K 47/18; A61P 37/00; C12N 15/70; C12N 1/20

(52) U.S. Cl. CPC A61K 38/164; C07K 14/35; A61K 9/06; A61K 47/02; C12N 1/20; A61K 47/10; A61K 47/183; A61P 37/00; C12N 15/70; A61K 47/12

(57) ABSTRACT

The present Invention provides a therapeutic composition for the treatment of Inflammation/tissue Injury and associated disorders. Particularly, the Invention provides a composition based on recombinantly purified PPE2 protein and its method of preparation.

This application claims the benefit of Indian Patent Application No. 201941000876, filed on Jan. 8, 2019, the disclosure of which is incorporated herein by reference.

### REFERENCE TO SEQUENCE LISTING SUBMITTED ELECTRONICALLY

A sequence listing electronically submitted with the present application as an ASCII text file named “SubstituteSequenceListing.TXT” created on Mar. 19, 2020 and having a size of 6 kilobytes, is incorporated herein by reference in its entirety.

### FIELD OF INVENTION

The present Invention provides a therapeutic composition for the treatment of Inflammation/tissue Injury and associated disorders. Particularly, the Invention provides a composition based on recombinantly purified PPE2 protein and its method of preparation.

### BACKGROUND OF THE INVENTION

The following background discussion includes information that may be useful in understanding the present invention. It is not an admission that any of the information provided herein is prior art or relevant to the presently claimed invention, or that any publication specifically or implicitly referenced is prior art.

Inflammation is a generic protective biological response towards a harmful stimulus (pathogens, damaged cells, or irritants) involving various molecular mediators and immune cells. It helps to eliminate the initial cause of cell injury and clears out necrotic cells and tissues. Classical signs of inflammation include edema, pain, redness and sometimes, organ mobility. It is an arm of innate immune response, mounted when host is exposed to a pathogen, tissue injury or an irritant (chemical/environmental) Inflammation results in vasodilatation followed by immigration of leukocytes and plasma from blood into the injured tissues. Inflammatory responses intended to be helpful to the body in circumventing pathological conditions Inflammation, also helps in tissue restoration in case of tissue injury caused by physical/chemical or environmental factors.

In general, the local inflammatory effects are beneficial, but at other times, it might be harmful to the body. Sustained or prolonged inflammation, also known as chronic inflammation, leads to higher immigration of immune cells followed by the destruction of tissues resulting into a pathological condition. Some of the classical examples of chronic inflammation are rheumatoid arthritis, periodontitis, atherosclerosis, and even cancer (e.g., gallbladder carcinoma). Humans as well as farm and pet animals like cows, buffalo, horses, cats and dogs also suffer from health problems associated with inflammation. Racehorses are prone to soft tissue injuries, which impart lameness and make them unfit for athletic activity. Sometimes, traumatic injuries induced inflammation may result into complete loss of the limbs. Similarly, house pets like cats and dogs are also vulnerable to inflammation and related disorders. Osteoarthritis is a common disease in pets, especially in dogs, which leads to permanent damage of joints resulting in lameness. In addition, inflammatory bowel disease (IBD) and physical tissue injury induced inflammation are frequent disorders commonly found in pet animals.

There are various factors i.e. TNF-α, TGF-β, which are reported to have important and decisive role in regulating the inflammatory response during acute and chronic inflammation (Brito et al., 1999). Host-response towards an infection is completely different than responses towards acute or chronic inflammation. In case of tissue damage, there is a surge of pro-inflammatory molecules in injured region. Non-Steroidal Anti-Inflammatory drugs (NSAIDs) are most preferred class of anti-inflammatory drugs. More than 90 percent of these NSAIDs are cyclooxygenase (Cox) inhibitors. In human and animals, steroids and non-steroidal anti-inflammatory drugs (NSAIDs) are the major class of drugs use for treatment of inflammatory conditions. Steroids, mainly glucocorticoids, represent the standard therapy for various chronic inflammatory diseases in humans such as asthma, rheumatoid arthritis (RA), inflammatory bowel disease and autoimmune diseases. Glucocorticoids act via binding with glucocorticoid receptors resulting in transcription of genes coding anti-inflammatory factors e.g. IL-10, IL-1 etc (Barnes P J et al., 1988),In case of humans, NSAIDs, e.g. Aspirin, Diclophenac, are another class of commonly used drugs, which are used to treat inflammation or inflammatory diseased condition. In case of animals, FDA has approved few NSAIDs to control pain and inflammation associated with osteoarthritis and tissue injury and inflammation after soft tissue and orthopedic surgery. Commonly used NSAIDs for the veterinary treatment for pets and other animals include Carprofen, Deracoxib, Firocoxib, Grapiprant and Meloxicam etc. NSAIDs act via blocking cyclo-oxygenase (COX) activity and thus inhibit prostaglandin synthesis which further brings down the inflammation.

However, it is seen that the prolonged usage of steroids is associated with various harmful side-effects involving major organ systems such as musculoskeletal, gastrointestinal, cardiovascular, endocrine, neuropsychiatric, dermatologic, ocular, and immunologic etc. (Oray M et al., 2016) and thus the steroids have disadvantages for prolonged use in humans. Recent studies have highlighted lots of side-effects of prolonged NSAIDs usage which includes nephrotoxicity (Pazhayattil G S et al., 2014), cardiovascular toxicity (El-Yazbi A F et al., 2017), gastrointestinal damage (Russell R I et al., 2001) myocardial infractions, blood thinning (Park K et al., 2013).

For animals, there are only handful of FDA approved anti-inflammatory drugs available in the market and most of them are not prescribed for more than three consecutive days. For instance, till date, there are only two FDA approved NSAIDs (meloxicam and robenacoxib) available for treatments of cats and meloxicam is permitted as a one-time injection only (FDA—https://www.fda.gov/AnimalVeterinary). In spite of such restricted use, NSAIDs impart adverse effects. Some of the most common side-effects of NSAIDs are vomiting, loss of appetite, low activity level, diarrhea. In horses, NSAIDs produces adverse gastrointestinal abnormalities including right dorsal colitis and inhibition of mucosal barrier healing (Marshall J F et al., 2011). Other side effects include both peptic and intestinal ulcers, stomach and intestinal perforations, kidney malfunction and failure, liver failure, and sometimes death. Therefore, there is a need of a therapeutic composition with broad-spectrum therapy having enhanced therapeutic effect for inflammatory conditions.

Now a days, protein and peptide-based drugs are in high demands due to specificity and lesser adverse effects than already existing small molecules-based drugs. Therefore, recombinantly purified protein/s are considered as a potent candidate for the development of therapeutic agents against inflammation and related disorders.

Proteins are biomolecules with specific 3-dimensional geometry which enable them to act either as enzymes, hormones, interferons or antibodies with high competency and accuracy. Therefore, therapeutic protein drugs are emerging as an important class of medicines in novel therapies. Recently, FDA has approved large number of recombinant protein therapeutics to treat clinical problems including autoimmunity/inflammation, infection, cancer and genetic disorders (Lagas se H A et al., 2017). In comparison with typical small-molecule drugs, proteins are highly specific and thus, may result in decreased toxicity and other adverse side-effects (Craik D J et al., 2013). The market on recombinant protein-based therapeutics is growing much faster than that of small molecules and will make up an even larger proportion of the market in near future. Hence, there is a need of a therapeutic composition with enhanced therapeutic efficacy and minimum side effects.

### OBJECT/S OF THE INVENTION

A primary object of the present invention is to overcome the drawbacks associated with the prior art.

Another object of this invention is to provide a therapeutic composition capable of effectively reducing inflammation.

Yet another object of the Invention is to provide a therapeutic composition comprising recombinantly purified PPE2 protein.

Yet another object of the Invention is to provide a therapeutic composition comprising recombinantly purified PPE2 protein as the active agent, additional agents and vehicle.

Yet another object of the Invention is to provide a method of preparing the therapeutic composition comprising recombinant PPE2 (rPPE2).

### BRIEF DESCRIPTION OF DRAWINGS

To further clarify advantages and features of the present invention, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings in which:

FIG. 1 (a) illustrates the rPPE2 reduces paw edema induced by formalin in mice at early time points, and FIG. 1 (b) illustrates results when Formalin-injected (0.02 ml of 5% in right hind paw) BALB/c mice were treated with Diclophenac or different doses of rPPE2 via intra-peritoneal route one hour (hr) post injection of formalin. Equal volume of PBS was injected in the left hind paw as vehicle control. Mice were examined for paw thickness for three hour post-treatment of PPE2/Diclophenac. Graphical representation of percentage inflammation in right hind paw of various groups. Data shown are mean±SD of 3 mice per group. rPPE2 treated groups were compared with PBS injected control group (two-way ANOVA with Bonferroni post hoc tests. (NS=non-significant)

FIG. 2 illustrates the rPPE2 treatment reduces the inflammation and tissue damage in paw samples.

FIG. 3 illustrates the rPEE2 treatment reduces mast cell population in inflamed paw tissues.

FIG. 4 illustrates the rPPE2 decreases formalin-induced paw edema during late time points.

FIG. 5 illustrates the Body weights for 21 days post treatment.

### DETAILED DESCRIPTION OF THE INVENTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof. Throughout the patent specification, a convention employed is that in the appended drawings, like numerals denote like components.

Reference throughout this specification to “an embodiment, “another embodiment, “an implementation, “another implementation” or similar language means that a particular feature, structure, or characteristic described in connection with the embodiment is included in at least one embodiment of the present invention. Thus, appearances of the phrase “in an embodiment, “in another embodiment, “in one implementation, “in another implementation, and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures proceeded by “comprises . . . a” does not, without more constraints, preclude the existence of other devices or other sub-systems or other elements or other structures or additional devices or additional sub-systems or additional elements or additional structures.

In an aspect, the Invention provides recombinant PPE2 protein.

In an embodiment, the recombinant PPE2 protein comprises SEQ ID NO:1. The protein with SEQ ID NO:1 comprises molecular weight of 57 kDa having a nuclear localization domain ranging from 469 to 480 amino acid and a DNA binding domain ranging from 319 to 340 amino acid.

The Invention in another aspect provides a method of purification of recombinant PPE2 protein as mentioned above. The method comprises following steps:
a) Cloning ORF comprising PPE2 into pRSET-A vector with 6X His- tag at its N-terminus to obtain PPE2-pRSET-A;
b) Transforming the construct PPE2-pRSET-A resulting from step (a) into Escherichia coli where said Escherichia coli comprises BL21 (DE3) pLYS strain where the positive transformants were inoculated into primary culture followed by secondary culture;
c) Inducing secondary culture with Isopropyl β-d-1 -thiogalactopyranoside at 37° C. for 4 to 5 hours followed by harvesting the bacteria pellets through centrifugation and lysing in phosphate buffer saline containing 5% glycerol;
d) Passing the lysate through column comprising TALON beads;
e) Washing the column with phosphate buffer saline comprising 30% imidazole;
f) Eluting PPE2 with 300 mM imidazole where said eluted protein is dialyzed followed by incubating with polymyxin B-Agarose beads to remove the residual bacterial endotoxin.

In an embodiment, the purification is performed through affinity chromatography.

In another aspect of the Invention, there is provided a pharmaceutical composition comprising recombinant PPE2 protein with SEQ ID NO:1.

In an embodiment, the pharmaceutical composition comprises recombinant PPE2 protein and a Buffer.

In an embodiment, the recombinant PPE2 protein is present in an amount ranging from 0.5 mg/kg to 3 mg/kg while said Buffer is present in an amount ranging from 300 μl-400 μl.

In an embodiment, the buffer comprises PBS, TBS.

In an embodiment, the composition is an anti-inflammatory, wound healing composition and associated disorders.

In an embodiment, the composition is in injectable form, gel form.

In an embodiment, the gel form of said composition is prepared by dissolving 1 mg recombinant PPE2 in approximately 5 gram of a gel composition.

In an embodiment, the gel composition comprises sodium CMC, sodium chloride, sodium acetate trihydrate, glacial acetic acid, methyl parahydroxy benzoate, propyl parahydroxy benzoate, m-cresol 99%, lysine hydrochloride, water.

In an embodiment of the Invention, the recombinantly purified PPE2 protein is present in the range of 0.5 mg/kg to 3 mg/kg dissolved in Phosphate buffer saline (PBS).

In an embodiment, the gel composition comprises following components:
a) sodium CMC (3% w/v)
b) sodium chloride (0.19%)
c) sodium acetate trihydrate (0.2%)
d) glacial acetic acid (0.1)
e) methyl parahydroxy benzoate (0.156%)
f) propyl parahydroxy benzoate (0.176%)
g) m-cresol (0.2%)
h) lysine hydrochloride (0.15%) and water.

The Invention performs the comparative experiment using Diclofenac sodium, a potent NSAID, as a standard control. It is observed that recombinantly purified PPE2 caused higher reduction in the edema and inflammation in both early and later time points as compared to Diclophenac (standard control). The therapeutic composition comprises PPE2 with dosage of almost one third of the standard drug. The composition shows better recovery.

### Method

1. Animals: Female BALB/c mice of 6-8 week of age were maintained at the animal house facility of Vimta Labs Ltd, Hyderabad and the experimental protocols were approved by and performed as per the guidelines of the Institutional Animal Ethics Committee (IAEC) of Vimta Labs Ltd, Hyderabad.
2. Induction of inflammation/tissue injury: A sub-planter injection of 0.02 ml of 5% formalin (Sigma Aldrich, USA) was administered to right hind paw. Same volume of PBS as vehicle control was injected into left hind paw. Induction of inflammation was confirmed by swelling in right hind paw using vernier caliper at various time points. Changes in body weights were recorded at various time points using an automatic electronic balance (Shimadzu, USA).
3. Administration of Drug or PPE2 protein: Animals were allocated to 5 groups with 7 mice per group. Various groups are 1) PBS control, 2) Diclophenac sodium treated (10 mg/kg), 3-5) rPPE2 treated (0.5, 1, 2 or 3 mg/kg). Diclophenac or PPE2 was administered via intra-peritoneal route after 1 hour of formalin injection.
4. Paw thickness measurements: Paw thickness was measured using electronic digital vernier caliper (Aerospace, India) and were recorded at various time points. Percentage of Inflammation was calculated using the following formula:

$$\frac{Post drug treated ( Right hind paw - Left hind paw )}{Pre drug treated ( Right hind paw - Left hind paw )} \times 100$$

5. Histopathology: Mice were sacrificed and paw samples were collected for all the groups and were fixed in 10% neutral buffered formalin followed by decalcification in 24.4% formic acid, and 0.5 Nsodium hydroxide for 5 days. Tissue samples were embedded in paraffin wax, sectioned (3˜5 μm) and stained with Toluidine blue and H&E (hematoxylin and eosin) dyes for estimation of mast cell population and infiltration of cells respectively.
6. Statistical analysis: Data were expressed as mean±SD and one-way ANOVA test (Bonferroni's post hoc test) was used to determine statistical differences between the groups and p<0.05 was considered to be significant. GraphPad Prism 5.02 software was used for statistical analysis.

The Invention is described with the help of non-limiting examples:

### EXAMPLE 1

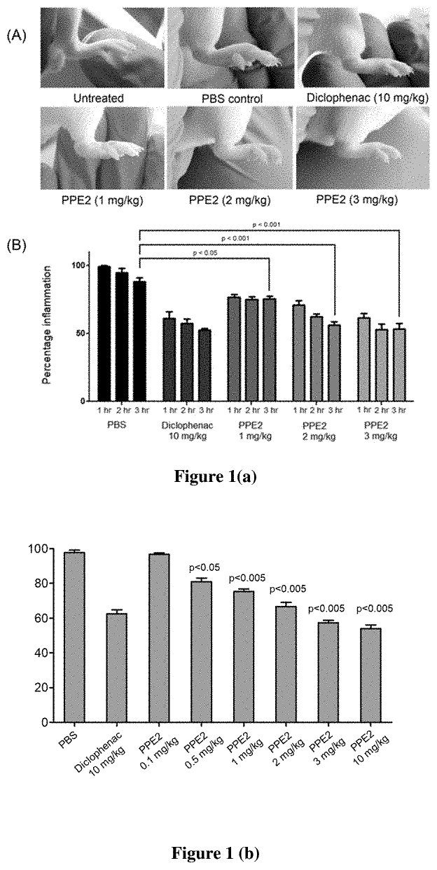

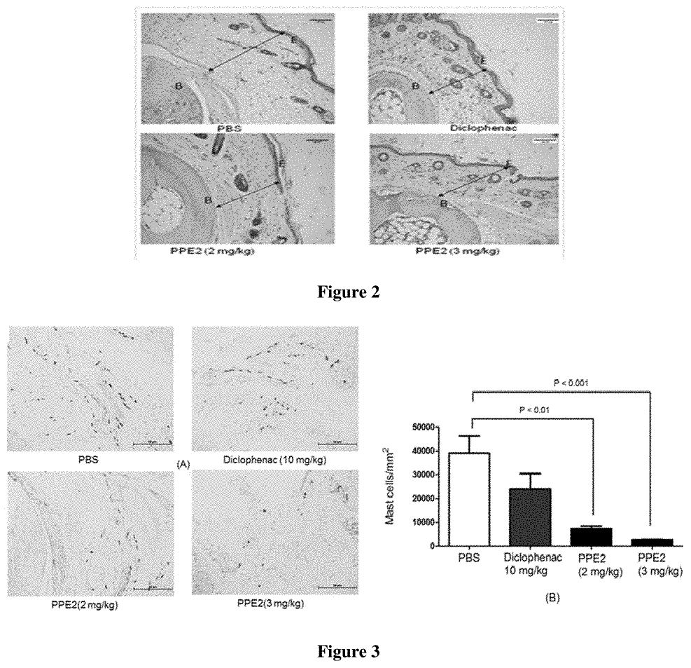

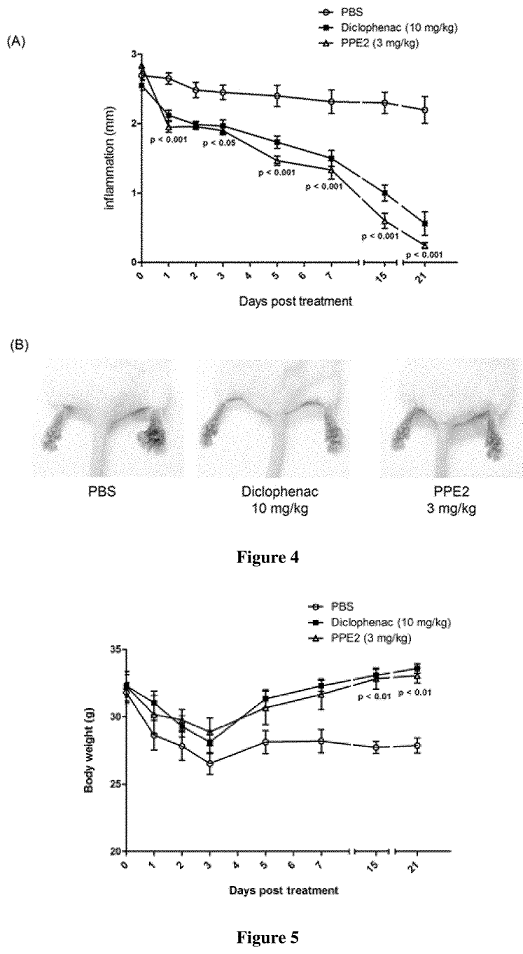

About 0.02 ml of 5% formalin was injected via sub-planter route into the right hind paw of BALB/c mice of 6-8 week age group. This experiment studies tissue inflammation, body weight, mast cell profiles and histopathology as a symptom of tissue injury induced by formalin. After the development of complete symptom, i.e. paw edema, rPPE2 was administered through intra-peritoneal route in four different doses i.e. 0.5 mg/kg, 1 mg/kg or 2 mg/kg or 3 mg/kg of body weight. Diclophenac sodium (10 mg/kg) was used as control drug and was administered via intra-peritoneal route. After treatments, for each group, body weights were taken and paw edema was measured using vernier caliper. It was found that mouse treated with different doses of rPPE2 showed reduction in the paw edema as compared to PBS/vehicle control and the best effect was obtained with 3 mg/kg concentration of rPPE2 for early time points (FIGS. 1A and 1B). Histopathological analysis of the paw tissues showed lesser tissue damage, reduced edema and infiltration of inflammatory cells after 3 hour of rPPE2 treatment (FIG. 2), which indicated that rPPE2 has anti-inflammatory activity. Toluidine blue staining of the same tissue samples showed a significant reduction in the mast cell population in formalin-injected mice treated with rPPE2 when compared to PBS control (FIGS. 3A and 3B) which might be the major factor in the reduction of inflammation in paw tissues. When all the groups were observed for paw thickness, it was seen that the rPPE2 (3 mg/kg) showed gradual and significant reduction in edema from day 5 and almost recovered in 21 days as compared to PBS control group (FIG. 4A). After 21 Days, it was observed that single dose of rPPE2 (3 mg/kg) showed almost complete abduction of inflammation and its symptoms when compared with Diclophenac and PBS control group (FIG. 4B) which indicate that along with anti-inflammatory activity, rPPE2 has a tissue protective activity as well. Whereas, the PBS control group sustained inflammation which led to severe damage of paw and surrounding tissues (FIG. 4B). During these 21 days, rPPE2 (3 mg/kg) treated mouse showed no change in body weight as compared to the Diclophenac group, though PBS group has shown a small dip in the body weight due to sustained inflammation (FIG. 5).

FIG. 5 describes the rPPE2 reduces paw edema induced by formalin in mice at early time points when BALB/c mice were injected with 0.02 ml of 5% formalin via sub-planter route into the right hind paw and were treated with either Diclophenac or different doses of rPPE2 via intra-peritoneal route one hour after injection of formalin. Same volume of PBS was injected in the right hind paw as vehicle control. Mice were examined for paw thickness for three consecutive hour post-treatment of PPE2/Diclophenac. (A) Representative photographs of right hindpaw after three hours of post-treatment. (B) Graphical representation of percentage inflammation in right hind paw of various groups. Data shown are mean±SD of 7 mice per group. rPPE2 treated groups were compared with PBS injected control group (two-way ANOVA with Bonferroni post hoc tests)

### EXAMPLE 2

Formalin-injected (0.02 ml of 5% in right hind paw) BALB/c mice were treated with Diclophenac or different doses of rPPE2 via intra-peritoneal route one hour post injection of formalin. Equal volume of PBS was injected in the left hind paw as vehicle control. Three hours post-treatment, mice were sacrificed and the paw sections were prepared and stained with hematoxylin and eosin. Photographs of representative sections were visualized at 40X magnification. The arrow represents thickness/edema (B=bone; E=epidermis). FIG. 6 describes that rPPE2 treatment reduces the inflammation and tissue damage in paw samples.

### EXAMPLE 3

Formalin-injected (0.02 ml of 5% in right hind paw) BALB/c mice were treated with Diclophenac or two different doses of rPPE2 via intra-peritoneal route, one hour post injection of formalin. Equal volume of PBS was injected in the left hind paw as vehicle control. Three hours post-treatment, mice were sacrificed and the paw sections were prepared and stained with toluidine blue to quantify mast cell population. (A) Photographs of representative sections were visualized at 40× magnification. (B) Counting of mast cells was performed in toluidine blue stained paw sections and were normalized per unit area (mm²). Data shown are mean±SD of 5 mice per group. rPPE2 treated groups were compared with PBS injected control group (one-way ANOVA with Bonferroni). FIG. 7 shows that rPEE2 treatment reduces mast cell population in inflamed paw tissues.

### EXAMPLE 4

FIG. 8 shows that rPPE2 decreases formalin-induced paw edema during late time points. The Formalin-injected (0.02 ml of 5% in right hind paw) BALB/c mice were treated with Diclophenacor rPPE2 (3 mg/kg) via intra-peritoneal route one hour post injection of formalin. Equal volume of PBS was injected in the left hind paw as vehicle control. (A) Mice were observed for paw thickness for a period of 21 days post-treatment of PPE2/Diclophenac Inflammation (mm) was calculated by subtracting thickness of left hind paw from right hind paw. Data shown are mean±SD of 5 mice per group.rPPE2 treated groups were compared with PBS injected control groups (two-way ANOVA with Bonferroni post hoc tests). (B) Representative photographs of right hind paw after 21 days of treatment.

### EXAMPLE 5

FIG. 5 shows about Body weights for 21 days post treatment. The Formalin-injected (0.02 ml of 5% in right hind paw) BALB/c mice were treated with Diclophenac or rPPE2 (3 mg/kg) via intra-peritoneal route one hour post injection of formalin. Equal volume of PBS was injected in the left hind paw as vehicle control and body weights were measured for 21 days. Data shown are mean±SD of 5 mice per group. rPPE2 treated groups were compared with PBS injected control groups (two-way ANOVA with Bonferroni post hoc tests).

We claim:

1. A recombinant PPE2 protein comprising SEQ ID NO:1, wherein said protein comprises molecular weight of 57 kDa having a nuclear localization domain ranging from 469 to 480 amino acid and a DNA binding domain ranging from 319 to 340 amino acid.

2. A method of purification of recombinant PPE2 protein as claimed in claim 1, comprising the steps of:
a) Cloning ORF comprising PPE2 into pRSET-A vector with 6X His- tag at its N-terminus to obtain PPE2-pRSET-A;
b) Transforming the construct PPE2-pRSET-A resulting from step (a) into Escherichia coli where said Escherichia coli comprises BL21 (DE3) pLYS strain where the positive transformants were inoculated into primary culture followed by secondary culture;
c) Inducing secondary culture with Isopropyl β-d-1-thiogalactopyranoside at 37° C. for 4 to 5 hours followed by harvesting the bacteria pellets through centrifugation and lysing in phosphate buffer saline containing 5% glycerol;
d) Passing the lysate through column comprising TALON beads;
e) Washing the column with phosphate buffer saline comprising 30% imidazole; and
f) Eluting PPE2 with 300 mM imidazole where said eluted protein is dialyzed followed by incubating with polymyxin B-Agarose beads to remove the residual bacterial endotoxin.

3. The method of purification as claimed in claim 2, wherein said purification is performed through affinity chromatography.

4. A pharmaceutical composition comprising:
a) Recombination PPE2 protein as claimed in claim 1;
b) Buffer
wherein said Recombination PPE2 protein is present in an amount ranging from 0.5 to 3 mg/kg while said Buffer is present in an amount ranging from 300 μl-400 μl.

5. The composition as claimed in claim 4, wherein said buffer comprises PBS, TBS.

6. The composition as claimed in claim 4, wherein said composition is an anti-inflammatory and wound healing composition.

7. The composition as claimed in claim 4, wherein said composition is in injectable form, gel form.

8. The composition as claimed in claim 7, wherein the gel form of said composition is prepared by dissolving 1 mg recombinant PPE2 in approximately 5 gram of a gel composition.

9. The composition as claimed in claim 8, wherein the gel composition comprises:
i) sodium CMC (3% w/v)
j) sodium chloride (0.19%)
k) sodium acetate trihydrate (0.2%)
l) glacial acetic acid (0.1)
m) methyl parahydroxy benzoate (0.156%)
n) propyl parahydroxy benzoate (0.176%)
o) m-cresol (0.2%)
p) lysine hydrochloride (0.15%) and water.
